(19) United States
(12) Patent Application Publication
(10) Pub. No.: US 2006/0099157 A1
(43) Pub. Date: May 11, 2006

(54) Ether alcohols used as solvents and emulsifiers and dispersions containing said ether alcohols

(75) Inventor: Gerd Dahms, Duisburg (DE)

Correspondence Address: BIRCH STEWART KOLASCH & BIRCH, PO BOX 747, FALLS CHURCH, VA, 22040-0747, US

(73) Assignee: IFAC GmbH & Co. KG, Dusburg (DE)

(21) Appl. No.: 10/517,704

(22) PCT Filed: Jun. 11, 2003
(86) PCT No.: PCT/EP03/06140

§ 371 Date: Aug. 30, 2005

(30) Foreign Application Priority Data
Jun. 12, 2002 (DE) 102262012

(51) Int. Cl. A01N 25/10; A61K 8/86

(52) U.S. Cl. 424/59; 504/360

(57) ABSTRACT

Ether alcohols of the general formula (I)
R¹—O-[EO—]ₙ[PO—]ₘR² where
R¹ is C₁₋₄-alkyl,
R² is hydrogen or C₁₋₄-alkyl, n is on average 1 to 100, m is on average 0 to n/2 EO, PO are basic building blocks derived from ethylene oxide and propylene oxide, which may be present in any order if both building blocks are present, are used as solvents, solubility promoters or dispersion auxiliaries for organic compounds which are insoluble or insufficiently soluble in lipophilic and hydrophilic media, and as dispersion auxiliaries or continuous phase for (micro)pigment dispersions.

The invention relates to the use of ether alcohols as solvents or solubility promoters for organic compounds which are insoluble or insufficiently soluble in lipophilic and hydrophilic media, and as dispersant for (micro)pigments. The invention also relates to ether alcohol/polyol-in-oil emulsions (EP/O emulsions) comprising ether alcohols, to methods for their preparation, to ether alcohol/polyol-in-oil-in-water emulsions (EP/O/W emulsions) comprising these emulsions, to the use thereof in cosmetic and/or pharmaceutical and/or agrochemical active ingredient compositions, and to such cosmetic and/or pharmaceutical and/or agrochemical active ingredient compositions.

In cosmetic and/or pharmaceutical and/or agrochemical active ingredient compositions, use is often made of emulsions containing active ingredients which can be used to prepare compositions suitable for the desired application form. The preparation of polyol-in-oil emulsions and polyol-in-oil-in-water emulsions is known per se. Using such emulsions it is possible to convert cosmetic and/or pharmaceutical active ingredients into a form suitable for the application. DE-A-43 431 114 relates to anhydrous X/O emulsions which comprise a nonaqueous, oil-immiscible phase and an oil phase. An active ingredient, which may also be in the form of a solid, for, for example, medicinal, cosmetic or technical applications may be comprised in the nonaqueous, oil-immiscible phase. The emulsion is prepared using a special emulsifier which has an HLB value of at most 6 or is a W/O emulsifier. As polyol which can be used, mention is made of propylene glycol, butylene glycol, polyalkylene glycol, glycerol, polyglycerol and mixtures thereof.

DE-A-43 31 113 relates to a stable multiple emulsion of the X/O/Y type. Here, X is an oil-immiscible component, 0 is an oil phase and Y is an aqueous phase.

W/O/W emulsions and P/O/W emulsions in particular are described where the polyol phase which can be used is again propylene glycol, butylene glycol, polyalkylene glycol, glycerol, polyglycerol or mixtures thereof.

A large number of cosmetic or pharmaceutical or agrochemical active ingredients are not soluble in hydrophilic and lipophilic media. In particular, they are not soluble in water or oil. Such active ingredients can therefore often only be applied in the form of finely divided dispersions of the solids in carrier media, which leads to disadvantages during formulation, metering and application. The presence of solids results in complex formulations of the cosmetic or pharmaceutical compositions being obtained. In addition, active ingredients can often not be supplied for an application in a suitable form.

An object of the present invention is to provide cosmetically, pharmaceutically and agrotechnically compatible solubility promoters for active ingredients which are insoluble or insufficiently soluble in hydrophilic and lipophilic media. In addition, the intention is to provide polyol-in-oil emulsions with a new type of polyol substitute component, where the substitute component can act as solubility promoter for the active ingredients. The disadvantages of the known active ingredient formulations should thus be overcome.

According to the invention, the object is achieved through the use of ether alcohols of the general formula (I)
R′—O-[EO—]ₙ[PO—]ₘR² (I)
where
R¹ is C₁₋₄-alkyl,
R² is hydrogen or C₁₋₄-alkyl,
n is on average 7 to 50, generally 1 to 100,
m is on average 0 to n/2
EO, PO are basic building blocks derived from ethylene oxide and propylene oxide, which may be present in any order if both building blocks are present,
   as solvents, solubility promoters or dispersion auxiliaries for organic compounds which are insoluble or insufficiently or not very soluble in lipophilic and hydrophilic media, and as dispersion auxiliaries or continuous phase for (micro)pigment dispersions and the resulting solutions and dispersions. The object is also achieved by an ether alcohol/polyol-in-oil emulsion comprising at least one ether alcohol of the general formula (I)
   R¹—O-[EO—]ₙ[PO—]ₘR² (I)
   where
R¹ is C₁₋₄-alkyl,
R¹ is hydrogen or C₁₋₄-alkyl,
n is on average 1 to 100,
m is on average 0 to n/2
EO, PO are basic building blocks derived from ethylene oxide and propylene oxide, which may be present in any order if both building blocks are present,
   in an oil-immiscible ether alcohol/polyol phase, an oil phase and at least one emulsifier.

In the ether alcohols of the general formula (I), basic building blocks derived from ethylene oxide and, if appropriate, additionally from propylene oxide may be present. These basic building blocks have the structures —CH₂—CH₂—O—, —CH₂—CH(CH₃)—O— and —CH(CH₃)—CH₂—O—. If both basic building blocks derived from ethylene oxide and propylene oxide are present, they may be in any order. This means that in each case one or more blocks derived from ethylene oxide and propylene oxide can be joined together. In addition, the units derived from ethylene oxide and propylene oxide may also be present alternately or randomly. The continuous transitions possible between these forms are likewise possible according to the invention.

In the general formula (I), the fraction of basic building blocks derived from propylene oxide is at most a fraction of the amount of basic building blocks derived from ethylene oxide. While, on average, 1 to 100, preferably 2 to 70, particularly preferably 3 to 50, especially 5 to 15, basic building blocks derived from ethylene oxide are present, 0 to n/2, preferably 0 to n/4, particularly preferably 0 to n/8, of basic building blocks derived from propylene oxide are on average present. If basic building blocks derived from propylene oxide are present, their amount is preferably n/10 to n/4, particularly preferably n/8 to n/5. The numbers n and m are average values since a distribution of the degree of alkoxylation is generally established during the alkoxylation. For this reason, fractional values for n and m are also possible. The width of the distribution of the degree of alkoxylation depends inter alia also on the alkoxylation catalyst used. It is also possible to establish discrete degrees of alkoxylation or very narrowly distributed distributions of the degree of alkoxylation.

The solutions and dispersions and the uses as solvent, solubility promoter or dispersion auxiliary, as given above, relate to ether alcohols of the general formula (I) in which n according to one embodiment is on average 7 to 50, preferably 7 to 15, in particular 8 to 15.

R¹ is a C₁₋₄-alkyl radical, preferably C₁₋₃-alkyl radical, particularly preferably C₁₋₂-alkyl radical, especially a methyl radical. Propyl radicals comprise n-propyl and isopropyl, while butyl radicals comprise n-butyl, isobutyl, tert-butyl.

R² is hydrogen or a radical as defined above for R¹. Here, the meaning of R² is independent of the meaning of the radical R¹. R² is particularly preferably hydrogen. The expression “ether alcohol” used in the description and the claims comprises all compounds of the general formula (I), i.e. also the cases in which R² is not a hydrogen atom and thus no free hydroxyl groups are present in the molecule.

The ether alcohol is preferably a methanol ethoxylate with 5 to 15, e.g. 5, 6, 7, 8, 9, 10, 11, 12, 13, 14 or 15, particularly preferably 7 to 13, ethylene oxide units.

Special preference is given to using polyethylene glycol monomethyl ether (12EO) and polyethylene glycol monomethyl ether (7EO) and 2-methoxyethanol, ethoxylated with 7EO. These are pure methyl alcohol ethoxylates. Such compounds are known per se and have hitherto been used for the preparation of terminally methyl-capped fatty acid polyethylene glycol esters. The compounds are commercially available.

According to the invention, particular preference is given to using ether alcohols of the general formula (I) which are liquid at room temperature (25° C.).

The expression “ether alcohol/polyol”, as is used for describing ether alcohol/polyol-in-oil emulsions (EP/O), ether alcohol/polyol phases and ether alcohol/polyol-in-oil-in-water emulsions (EP/O/W) indicates that an ether alcohol phase or a mixed ether alcohol/polyol phase may be present. The corresponding carrier phase is thus formed either only by the ether alcohols of the general formula (I), or there are mixtures of the ether alcohols with polyols present. In addition, it is possible, as described below, for organic or inorganic compounds to be present in the ether alcohol/polyol phase.

One or more ether alcohol/polyol phases may be present in the ether alcohol/polyol-in-oil emulsions according to the invention (EP/O emulsions). Preferably, an ether alcohol/polyol phase is present which comprises at least one ether alcohol as described above. Preferably, at least 50% by weight, particularly preferably at least 80% by weight, especially at least 95% by weight, of the ether alcohol is present in the polyol phase, based on the total ether alcohol/polyol phase. In the ether alcohol/polyol phase it is also possible for water to be comprised, in an amount of at most 20% by weight, preferably at most 10% by weight in particular at most 5% by weight, based on the total ether alcohol/polyol phase. The ether alcohol/polyol phase is preferably largely or completely anhydrous. The sum of ether alcohol, other polyol and water gives here 100% by weight.

As polyols, the customary known polyols, such as propylene glycol, butylene glycol, ethylene glycol, polyalkylene glycol, glycerol, polyglycerol, glycosides, sorbitol, mannitol, pentaerythritol, trimethylolpropane or mixtures thereof, may be present. Suitable polyalkylene glycols are, in particular polyethylene glycol and polypropylene glycol. Further suitable polyols are known to the person skilled in the art, for example aromatic polyols, such as emodin/aloe vera.

Particularly preferably, only ether alcohol is present in the ether alcohol/polyol phase, as is described above.

According to the invention, the ether alcohol/polyol phase is not miscible with oil. This means that during the preparation of the EP/O emulsions according to the invention, preferably no ether alcohol/polyol dissolves in the oil phase and no oil dissolves in the ether alcohol/polyol phase. Slight deviations from this (for example up to 5% by weight, preferably up to about 1% by weight, especially up to about 0.5% by weight solubility) are unimportant here. What is important is that an emulsion of the ether alcohol/polyol phase in the oil phase is obtained in the EP/O emulsion, and no solution of the ether alcohol/polyol in the oil.

Oils which may be used in the oil phase are all known suitable oils and mixtures thereof. Examples of suitable oils are silicone oils and derivatives thereof, which may be linear or cyclic, natural ester oils, such as grapeseed oil, olive oil or sunflower oil, synthetic ester oils, such as neutral oils, which may be linear or branched, paraffin oils and isoparaffin oils, ester oils, for example of citrates, lactates, aleates, salicylates, cinnamates or other organic light protection filters, or of camphor derivatives, triglycerides, fatty alcohols or mixtures thereof.

In the EP/O emulsions, the weight ratio of ether alcohol/polyol phase to oil phase is preferably 10:90 to 90:10, particularly preferably 25:75 to 75:25 and especially 40:60 to 60:40.

Emulsifiers which can be used are any suitable emulsifiers which are suitable for preparing an emulsion of the ether alcohol/polyols in oil. Examples of suitable emulsifiers are glycerol esters, polyglycerol esters, sorbitan esters, sorbitol esters, fatty alcohols, propylene glycol esters, alkyl glucoside esters, sugar esters, lecithin, silicone copolymers, wool wax and mixtures or derivatives thereof. Glycerol esters, polyglycerol esters, alkoxylates and fatty alcohols, and isoalcohols may be derived, for example, from castor fatty acid, 12-hydroxystearic acid, isostearic acid, oleic acid, linoleic acid, linolenic acid, stearic acid, myristic acid, lauric acid and capric acid. Besides said esters, succinates, amides or ethanolamides of fatty acids may also be present. Suitable fatty acid alkoxylates are in particular the ethoxylates, propoxylates or mixed ethoxylates/propoxylates.

The amount of emulsifier can be adapted to the practical requirements. Preferably, the emulsifier is used in an amount of from 0.1 to 20% by weight, particularly preferably 0.5 to 15% by weight, especially 1 to 8% by weight, based on the total EP/O emulsion. Occasionally, concentrations differing from these may be required.

According to one embodiment of the invention, the ether alcohol/polyol phase or the ether alcohol solution or dispersion may comprise a cosmetic and/or pharmaceutically active ingredient or perfume, perfume oils or aromas dissolved in the phase. This may also be a detergent, food or agriculture (agrochemical) active ingredient.

The active ingredients are preferably organic compounds which are insufficiently soluble, in particular insoluble, in lipophilic and hydrophilic media. In particular, the compounds here are insufficiently soluble or insoluble in water and oil. It is possible to use any suitable active ingredients provided they dissolve in the ether alcohol or the ether alcohol/polyol phase comprising the ether alcohol. Suitable active ingredients are, for example, dichlorfenac, ibuprofen, acetylsalicylic acid, salicylic acid, erythromycin, ketoprofen, cortisone, glucocorticoids.

Also suitable are cosmetic active ingredients which are, in particular, sensitive to oxidation or hydrolysis, such as, for example, polyphenols. Mention may be made here of catechins (such as epicatechin, epicatechin-3-gallate, epigallocatechin, epigallocatechin-3-gallate), flavonoids (such as luteolin, apigenin, rutin, quercitin, fisetin, kaempherol, rhametin), isoflavones (such as genistein, daidzein, glycitein, prunetin), cumarins (such as daphnetin, umbelliferone), emodin, resveratrol, oregonin.

Vitamins such as retinol, tocopherol, ascorbic acid, riboflavin, pyridoxine are suitable.

Also suitable are whole extracts from plants which comprise, inter alia, the above molecules or classes of molecule.

According to one embodiment of the invention, the active ingredients are light protection filters. Being organic-like protection filters, these may be present at room temperature (25° C.) in liquid or solid form. Suitable light protection filters (UV filters) are, for example, compounds based on benzophenone, diphenyl cyanoacrylate or p-aminobenzoic acid. Specific examples are (INCI or CTFA names) Benzophenone-3, Benzophenone-4, Benzophenone-2, Benzophenone-6, Benzophenone-9, Benzophenone-1, Benzophenone-11, Etocrylene, Octocrylene, PEG-25 PABA, Phenylbenzimidazole Sulfonic Acid, Ethylhexyl Methoxycinnamate, Ethylhexyl Dimethyl PABA, 4-Methylbenzylidene Camphor, Butyl Methoxydibenzoylmethane, Ethylhexyl Salicylate, Homosalate and methylenebisbenzotriazolyl tetramethylbutylphenol (2,2′-methylenebis {6-(2H-benzotriazol-2-yl)-4-(1,1,3,3-tetramethylbutyl)phenol}, 2-hydroxy-4-methoxybenzophenone-5-sulfonic acid and 2,4,6-trianilino-p-(carbo-2′-ethylhexyl-1′-oxi)-1,3,5-triazine.

Further organic light protection filters are octyltriazone, avobenzone, octyl methoxycinnamate, octyl salicylate, benzotriazoles and triazines.

The ether alcohols of the general formula (I) can also be used as solubility promoter and/or dispersant for antidandruff agents.

According to a further embodiment of the invention, the active ingredients used are antidandruff active ingredients as are customarily present in cosmetic or pharmaceutical formulations. One example is piroctone olamine (1-hydroxy-4-methyl-6-(2,4,4,-dimethylpentyl)-2(1H)-pyridone; preferably in combination with 2-aminoethanol (1:1)). Further suitable agents for the treatment of dandruff are known to the person skilled in the art.

According to a further embodiment of the invention, organic dyes are used as active ingredients or in place of active ingredients.

Any suitable amounts of the active ingredient can be incorporated into the ether alcohol/polyol phase. The amount to be incorporated is often determined by the solubility and by the ultimate field of application of the preparation. As a rule, 0.01 to 95% by weight, preferably 0.1 to 80% by weight, in particular 2 to 50% by weight, of the active ingredient are introduced into the ether alcohol or the ether alcohol/polyol phase, where the weight fraction relates to the sum of active ingredient and ether alcohol/polyol. Expressed in another way, in the amount of ether alcohol/polyol phase given above, the stated weight fraction of ether alcohol/polyol can be replaced by the active ingredient. For example, based on the pure ether alcohol/polyol phase, 0.01 to 95% by weight of the ether alcohol/polyol phase can be replaced by the active ingredient. This means that the above statements relating to the fraction of ether alcohol/polyol phase in the emulsion still apply. The content of ether alcohol/polyol or ether alcohol then decreases by precisely the fraction of active ingredient which is then present in the ether alcohol/polyol phase.

It is also possible to top up a customary carrier system for the active ingredient with the ether alcohol in order to arrive at said ether alcohol/polyol phase.

The above-described EP/O emulsion can also be emulsified in water or a water-in-oil emulsion. This results in an ether alcohol/polyol-in-oil-in-water emulsion (EP/O/W) emulsion which comprises at least one emulsion as described above and additionally at least one aqueous phase. Such multiple emulsions can correspond in structure to the emulsions described in DE-A-43 41 113, where the polyol component is varied in a manner according to the invention. The structure of the ether alcohol/polyol-in-oil emulsion can correspond to the structure of the emulsions described in DE-A-43 41 114, with the ether alcohol/polyol phase according to the invention being used as polyol phase.

When incorporating the EP/O emulsion according to the invention into water or aqueous systems, the weight ratio of the individual phases can be varied within wide ranges. Preferably, in the EP/O/W emulsion ultimately obtained, the weight fraction of the EP/O emulsion is 0.01 to 80% by weight, particularly preferably 0.1 to 70%, in particular 1 to 30% by weight, based on the total EP/O/W emulsion.

When incorporating the EP/O emulsion according to the invention into an O/W emulsion, the fraction of the EP/O emulsion is preferably 0.01 to 60% by weight, particularly preferably 0.1 to 40% by weight, in particular 1 to 30% by weight, based on the EP/O/W emulsion ultimately obtained. In the O/W emulsion which is used for this purpose, the oil fraction is preferably 1 to 80% by weight, particularly preferably 1 to 30% by weight, based on the O/W emulsion used.

The individual phases of the emulsions may also have customary ingredients known for the individual phases. For example, the individual phases can comprise further pharmaceutical or cosmetic active ingredients soluble in these phases. The aqueous phase may, for example, comprise organic soluble light protection filters, hydrophilically coated micropigment, electrolyte, alcohols etc. Some or all of the phases may also comprise solids, which are preferably chosen from pigments or micropigments, microspheres, silica gel and similar substances. The oil phase can, for example, comprise organically modified clay minerals, hydrophobically coated (micro)pigments, organic oil-soluble light protection filters, oil-soluble cosmetic active ingredients, waxes, metal soaps, such as magnesium stearate, vaseline or mixtures thereof. (Micro)pigments which may be specified are titanium dioxide, zinc oxide and barium sulfate, and wollastonite, kaolin, talc, Al₂O₃, bismuth oxychloride, micronized polyethylene, mica, ultramarine, eosine colors, azo dyes. In particular, titanium dioxide or zinc oxide are customary in cosmetics and light protection filters and can be applied to the skin in a particularly smooth and uniform way by means of the emulsions according to the invention. Microspheres or silica gel can be used as carrier for active ingredients, and waxes can, for example, be used as a basis for polishes.

The ether alcohols according to the invention can also be used as dispersion auxiliaries or continuous phase for (micro)pigment dispersions. Such systems of ether alcohols and (micro)pigments according to the invention typically comprise 10 to 50% by weight of (micro)pigments, particularly preferably 35 to 45% by weight of (micro)pigments. The dispersions obtainable from the (micro)pigments and the ether alcohols can be introduced into water or into oil without the dispersion being adversely affected. By using the ether alcohols, it is thus possible to hydrophobicize hydrophilic pigments and to incorporate them into an oil phase without the degree of dispersion being impaired. The invention thus also provides a dispersion of (micro)pigments and/or insoluble organic compounds in ether alcohols of the general formula (I).

The water phase can, moreover, comprise glycerol, polyethylene glycol, propylene glycol, ethylene glycol and similar compounds and derivatives thereof.

In a corresponding way, it is also possible to take medicaments which are administered orally, but are not stable under the conditions which prevail in the stomach or should start to take effect at other sites of the digestive tract to their site of activity using an emulsion according to the invention. This emulsion is not broken in the stomach, meaning that it passes the stomach unchanged. The active ingredient present is therefore released only subsequently in the digestive tract while the emulsifier is degraded or dissolved. The choice of emulsifiers can be made according to the conditions existing at the sites of activity.

The use of customary auxiliaries and additives in the emulsions is known to the person skilled in the art.

The EP/O emulsions according to the invention can be prepared by known processes, as are described, for example, in DE-A-43 41 114 and DE-A-43 41 113. For the preparation, the ether alcohol/polyol phase and the oil phase, each of which can comprise emulsifier, are heated separately to a temperature in the range from 20 to 90° C. and then combined with stirring.

Depending on the composition, on the phase volume ratio and the solids content which may be present, the emulsions can be prepared and exist in the form of solid or flowable emulsions. These are very stable emulsions which, under normal handling conditions, have a high long-term stability. In particular, they satisfy the usual stability requirements in the temperature range from −5° C. to +45° C. The droplets present in the emulsion are very stable, for which reason the emulsions are particularly suitable as carriers for many types of active ingredients.

The emulsions prepared using the specified emulsifiers can be obtained by a simple mixing operation with stirring, where the stability of the emulsions is generally barely influenced, if at all, by the stirrer energy introduced and the type of stirring tool. Any suitable standard commercial stirrer can be used to prepare the emulsion according to the invention.

The emulsions according to the invention are preferably used in cosmetic and/or pharmaceutical and/or agrochemical active ingredient compositions, e.g. sunscreen compositions/light protection compositions. The invention thus also provides such cosmetic and/or pharmaceutical and/or agrochemical active ingredient compositions which comprise at least one of the specified emulsions. The cosmetic and/or pharmaceutical compositions are hand or body lotions, oils, ointments, pastes, gels, lip care products, face care products and similar compositions. The compositions may be used in solid, liquid or aerosol form.

According to one embodiment of the invention, these are sunscreen compositions (light protection compositions) for application to the human skin. They can have the described solution/emulsion/dispersion forms. A suitable sunscreen composition comprises ether alcohols of the general formula (I), (micro)pigments of the given type and organic light protection filters, where at least the (micro)pigments are present in dispersed form. Suitable compositions comprise 10 to 80% by weight of ether alcohols of the general formula (I), 5 to 50% by weight of (specified) (micro)pigments, e.g. TiO₂ or ZnO, and 5 to 50% by weight of (specified) organic light protection filters, where the total weight of these ingredients is at most 100% by weight and customary further ingredients may be present. The ether alcohols have on average n=1 to 100, preferably 2 to 70, particularly preferably 3 to 50, in particular 5 to 15, or else 7 to 50, preferably 7 to 15, in particular 8 to 15.

The invention further provides the use of ether alcohols of the general formula (I) as solvent, solubility promoter or dispersion auxiliary for organic compounds which are sparingly soluble (<10% solubility) or insoluble in lipophilic and hydrophilic media. Suitable molecules or molecule groups are, for example, salicylic acid, sphingosines, ceramides, triterpenaric acid, such as oleanolic acid, betulinic acid, betulin, ursolic acid, boswellic acid, 18-β-glycyrrhetinic acid, forskolin, sclareolide, andrographolides.

Also suitable are whole extracts from plants which comprise, inter alia, the above molecules or classes of molecule.

Suitable lipophilic media here are the abovementioned oils, suitable hydrophilic media are aqueous media with a water fraction of at least 80% by weight, preferably at least 90% by weight, in particular at least 95% by weight. Compounds are regarded as being insoluble if their solubility is less than 3%, preferably less than 1%, in particular less than 0.5%.

The invention also relates to a solution of organic compounds that are insoluble in lipophilic and hydrophilic media in ether alcohols which have the structure as defined above.

In addition, the invention relates to the use of ether alcohols as solubility promoters for introducing cosmetic and/or pharmaceutical and/or agrochemical active ingredients into polyol-in-oil emulsions or polyol-in-oil-in-water emulsions.

The emulsions according to the invention can exhibit a transport and a depot effect for the active ingredients present therein. They can therefore act not only as carriers but also as depots which permit delayed release of the active ingredient over a certain period.

The emulsions according to the invention exhibit the following advantages:

Oxidation-sensitive and hydrolysis-sensitive active ingredients can be encapsulated in a manner which is stable and, in particular, storage-stable over a prolonged period. The moisture attack on the active ingredients can be reliably prevented by the emulsions according to the invention.

Active ingredients that are insoluble in hydrophilic and lipophilic media can be administered in the form of an emulsion or dispersion.

The penetration properties of the cosmetic or pharmaceutical compositions can be positively influenced through the use of the emulsions according to the invention. In particular, in the multiple emulsions described, a depot effect and improved penetration can be achieved.

The surmounting of the skin permeability barrier is achieved by, as a result of the formation of liquid-crystalline gel network structures in the described multiple emulsions, an order state being constructed which corresponds to that of the lipid barrier of the skin. In such a carrier system, the penetration properties of said pharmacological and cosmetic active ingredients are decisively improved.

The invention is explained in more detail by the examples below.

### EXAMPLE 1

Solution of Light Protection Agents

The light protection agent 2,4,6-trianilino-p-(carbo-2′-ethylhexyl-1′-oxy)-1,3,5-triazine was dissolved in polyethylene glycol monomethyl ether (7 EO) at room temperature with stirring. A solution was accessible which, based on the total solution, comprised 60% by weight of the light protection agent.

This 60% strength solution could be mixed in any mixing ratios into C₁₂-C₁₅-alcohol benzoate, the resulting system being stable in a broad temperature range from 5 to 50° C. At room temperature (25° C.), a storage time of more than 7 days was determined without resulting in flocculation or phase separation.

In customary oils, the solubility of this light protection agent was hitherto at most 13% (PPG-3 myristyl ether). In C₁₂-C₁₅-alcohol benzoate only a solubility of about 4% has hitherto been found.

### EXAMPLE 2

Solution of a Light Protection Agent

The light protection agent 2-hydroxy-4-methoxybenzophenone (benzophenone-3) could be dissolved at room temperature in polyethylene glycol monomethyl ether (7 EO), the fraction of light protection agent in the solution being more than 30% by weight.

Hitherto, only solutions which comprised at most 20% by weight of the light protection agent were accessible.

### EXAMPLE 3

Solubility of Light Protection Agents

2-Hydroxy-4-methoxybenzophenone-5-sulfonic acid could be dissolved in polyethylene glycol monomethyl ether (7 EO), the resulting solution comprising more than 10% by weight of the light protection agent. The per se water-soluble light protection agent could be introduced into an oil phase as polyethylene glycol monomethyl ether (7 EO) solution so that it could be used in this formulation as oil-soluble light protection filter.

### EXAMPLE 4

Example of the Preparation of an EP/O Emulsion

| | | |
|---|---|---|
| Trade name | CTFA/INCI | % by wt |

Phase A

| | | |
|---|---|---|
| Polyphenols | Polyphenols | 5.00 |
| Marlipal 1/7 | 2-methoxyethanol, | 60.0 |
| | ethoxylated w. 7 EO | |
| Dow Corning DC | Cyclomethicone, | 15.90 |
| 5225 Formulation | dimethicone copolyol | |
| Aid | | |
| Wacker Belsil→ | Cyclomethicone | 15.10 |
| CM 040 | | |
| Abil→EM 97 | Bis-PEG/PPG-14/14 | 4.00 |
| | Dimethicone | |
| Total | | 100.00 |

The emulsion was prepared by firstly preparing phases A and B separately. Phase A was then stirred slowly into phase B at room temperature until the average particle diameter in the emulsion was <500 nm. The particle size was determined by laser-light scattering (Fraunhofer diffraction).

### EXAMPLE 5

Example of the Preparation of an O/W Emulsion

| | | | |
|---|---|---|---|
| | | OW1 | OW2 |
| Trade name | CTFA/INCI | [% by wt] | [% by wt] |

Phase A

| | | | |
|---|---|---|---|
| Ceralution→H | Behenyl alcohol, | 4.00 | 4.00 |
| | glyceryl stearate, | | |
| | glyceryl stearate/ | | |
| | citrate, sodium | | |
| | dicocoylethylenediamine | | |
| | PEG-15 sulfate | | |
| Cosmacol→EBI | C₁₂₋₁₅ alkyl benzoate | 12.00 | 5.00 |
| Cosmacol→ELI | Di C₁₂₋₁₃ alkyl lactate | 3.00 | 0.00 |

Phase A1

| | | | |
|---|---|---|---|
| Marlipal→1/7 | 2-Methoxyethanol, | 9.00 | 5.00 |
| | ethoxylated w. 7 EO | | |
| Salicylic acid | Salicylic acid | 1.00 | 0.00 |
| Uvinul→150 | Octyl triazone | 0.00 | 5.00 |

Phase B

| | | | |
|---|---|---|---|
| Pur. water | Aqua | ad 100 | ad 100 |
| Keltrol→ | Xanthan gum | 0.25 | 0.25 |

Phase C

| | | | |
|---|---|---|---|
| Preservative | Preservative | q.s. | q.s. |
| Total | | 100.00 | 100.00 |

To prepare the O/W emulsion, the individual phases were firstly prepared separately. Phases A and B were then heated to 60° C. Phase A1 was heated to about 65° C. until all of the ingredients had dissolved. It was then slowly added to phase A. Phase B was then added to phases A/A1 and it was homogenized. The mixture was then cooled to 40° C. and homogenized further. After further cooling to 35° C., phase C was slowly introduced.

### EXAMPLE 6

Example of the Preparation of an EP/O/W Emulsion

| | | |
|---|---|---|
| | | EP/O/W |
| Trade name | CTFA/INCI | [% by wt] |

Phase A

| | | |
|---|---|---|
| Ceralotion→H | Behenyl alcohol, | 5.00 |
| | glyceryl stearate, | |
| | glyceryl stearate/ | |
| | citrate, sodium | |
| | dicocoylethylenediamine | |
| | PEG-15 sulfate | |
| Arlamol→HD | Isohexadecane | 5.00 |
| Belsil→CM40 | Cyclomethicone | 3.00 |

Phase B

| | | |
|---|---|---|
| Pur. water | Aqua | ad 100 |
| Keltrol→ | Xanthan gum | 0.3 |

Phase C

| | |
|---|---|
| EP/O emulsion | 10.00 |
| from example 4 | |

Phase D

| | | |
|---|---|---|
| Preservative | Preservative | q.s. |
| Total | | 100.00 |

To prepare the EP/O/W emulsion, the individual phases were firstly prepared separately. Phases A and B were then heated to 60° C., and phase A was slowly added to phase B. The mixture was then homogenized, cooled to 40° C. and further homogenized. Phase C was then added with stirring. The mixture was further stirred until the average multiple droplet size was 15 μm. The mixture was then cooled to 30° C., and phase D was slowly introduced.

The physical stability of the EP/O, O/W and EP/O/W emulsions is at least 3 months at 50° C. and more than 6 months at room temperature. The assessment was made visually and using a light microscope.

### EXAMPLE 7

Solution of Salicylic Acid

Salicylic acid was dissolved at room temperature in polyethylene glycol monomethyl ether (7 EO). A solution was accessible which comprised more than 10% by weight of salicylic acid in the acid form. The solution could be introduced in any fractions into C₁₂-C₁₅-alcohol benzoate without resulting in precipitation of the salicylic acid.

### EXAMPLE 8

Dispersion of Micropigments

Hydrophilically or hydrophobically coated titanium dioxide micropigments were introduced into polyethylene glycol monomethyl ether (7 EO). The hydrophilically coated titanium dioxide used was UV Titan M 212 (Kemira), and the hydrophobically coated titanium dioxide used was UV Titan M 262 (Kemira). In each case a dispersion was accessible which comprised 40% by weight of titanium dioxide in 60% by weight of polyethylene glycol monomethyl ether (7 EO). The dispersion had a better particle size distribution than standard commercial dispersions. The particle size distribution was determined by means of laser-light scattering (Fraunhofer diffraction). 100% of the particles had a diameter of less than 1 μm, with the average particle diameter being 350 nm.

Both dispersions could be dispersed in any ratios in water, during which the average particle diameter stayed the same. In addition, both dispersions could be introduced into C₁₂-C₁₅-alcohol benzoate in any ratios, during which the average particle size likewise stayed the same. The dispersion could be completely integrated into C₁₂-C₁₅-alcohol benzoate.

1-14. (canceled)

15. A solution of organic compounds which are insoluble or insufficiently soluble in lipophilic and hydrophilic media in ether alcohols of the general formula (I)
R¹—O-[EO—]ₙH (I)
where
R¹ is C₁₋₃-alkyl,
n is on average 7 to 15,
EO is building blocks derived from ethylene oxide.

16. A dispersion of (micro)pigments and/or insoluble organic compounds in ether alcohols of the general formula (I)
R′—O-[EO—]ₙH (I)
where
R¹ is C₁₋₃-alkyl,
n is on average 7 to 50,
EO is building blocks derived from ethylene oxide.

17. An ether alcohol/polyol-in-oil emulsion comprising at least one ether alcohol of the general formula (I)
R¹—O-[EO—]ₙH (I)
where
R¹ is C₁₋₃-alkyl,
n is on average 7 to 15,
EO is building blocks derived from ethylene oxide, in an oil-immiscible ether alcohol/polyol phase, an oil phase and at least one emulsifier.

18. The emulsion as claimed in claim 17, characterized in that, in the general formula (I), R¹ is C₁₋₂-alkyl, and n is on average 8 to 15.

19. The emulsion as claimed in claim 17, characterized in that the ether alcohol is a methanol ethoxylate with 7 to 13 ethylene oxide units.

20. The emulsion as claimed in claim 17, characterized in that at least 50% by weight of ether alcohol is present in the ether alcohol/polyol phase.

21. The emulsion as claimed in claim 17, characterized in that the ether alcohol/polyol phase comprises a cosmetic and/or pharmaceutical and/or agrochemical active ingredient dissolved in the phase.

22. A method of preparing emulsions as claimed in claim 17, characterized in that the ether alcohol/polyol phase and the oil phase, which may each comprise emulsifier, are heated separately to a temperature in the range from 20 to 90° C. and then combined with stirring.

23. An ether alcohol/polyol-in-oil-water emulsion comprising at least one emulsion as claimed in claim 17 and additionally at least one aqueous phase.

24. A cosmetic and/or pharmaceutical and/or agrochemical active ingredient composition comprising at least one emulsion as claimed in claim 17.

25. A sunscreen composition comprising
10 to 80% by weight of at least one ether alcohol, as is defined in claim 17,
5 to 50% by weight of at least one (micro)pigment,
5 to 50% by weight of at least one organic light protection filter and, if appropriate, further customary ingredients, where the total weight is 100% by weight.
